(19) United States
(12) Patent Application Publication
(10) Pub. No.: US 2021/0052149 A1
(43) Pub. Date: Feb. 25, 2021

(54) IMAGING APPARATUS

(71) Applicant: CANON KABUSHIKI KAISHA, Tokyo (JP)

(72) Inventors: Junya Fukumoto, Yokohama-shi (JP); Yuma Kudo, Kawasaki-shi (JP)

(21) Appl. No.: 16/987,600

(22) Filed: Aug. 7, 2020

(30) Foreign Application Priority Data
Aug. 22, 2019 (JP) 2019-151776

(51) Int. Cl. A61B 1/06; H04N 5/33; H04N 5/235; A61B 1/00; A61B 1/04

(52) U.S. Cl. CPC A61B 1/0638; H04N 5/33; A61B 1/042; A61B 1/00009; H04N 5/2354

(57) ABSTRACT

An imaging apparatus includes an optical element configured to separate incident light into light components in at least three types of wavelength bands, and a plurality of imaging elements configured to receive the light components in the at least three types of wavelength bands separated by the optical element, respectively. At least one imaging element of the plurality of imaging elements has a function of further separating a wavelength band of light that has entered the imaging element into at least two types of wavelength bands.

### BACKGROUND OF THE INVENTION

### Field of the Invention

The present disclosure relates to an imaging apparatus.

### Description of the Related Art

An imaging apparatus, for example, an endoscopic apparatus has conventionally mainly used a CCD (Charge Coupled Apparatus) image sensor. Recently, however, a CMOS (Complementary Metal Oxide Semiconductor) image senor is mainly used because of its advantages such as low cost, single power supply, and low power consumption. As the CMOS image sensor, a rolling shutter method is often employed in general (see Japanese Patent Laid-Open No. 2018-175871).

### SUMMARY OF THE INVENTION

One of problems to be solved by an embodiment disclosed in this specification is to ensure image quality sufficiently for observation. However, the problem is not limited to this, and obtaining functions and effects derived by configurations shown in an embodiment configured to implement the present invention to be described later can also be defined as another problem to be solved by the embodiment disclosed in this specification and the like.

An imaging apparatus according to an embodiment is an imaging apparatus comprising: an optical element configured to separate incident light into light components in at least three types of wavelength bands; and a plurality of imaging elements configured to receive the light components in the at least three types of wavelength bands separated by the optical element, respectively, wherein at least one imaging element of the plurality of imaging elements has a function of further separating a wavelength band of light that has entered the imaging element into at least two types of wavelength bands.

Further features of the present invention will become apparent from the following description of exemplary embodiments (with reference to the attached drawings).

### BRIEF DESCRIPTION OF THE DRAWINGS

FIG. 1 is a block diagram showing an example of the configuration of an imaging system including an imaging apparatus according to an embodiment;

FIG. 2 is a view showing a part of the configuration of the imaging apparatus according to an embodiment;

FIG. 3 is a view showing an example of a filter according to an embodiment;

FIG. 4 is a view showing an example of the imaging operation of an imaging apparatus according to a comparative example;

FIG. 5 is a view for explaining a problem of the imaging apparatus according to the comparative example;

FIG. 6 is a view showing an example of the imaging operation of the imaging apparatus according to an embodiment;

FIG. 7 is a view for explaining pixel interpolation processing of the imaging apparatus according to an embodiment;

FIG. 8 is a view showing an example of the imaging operation of an imaging apparatus according to an first modification;

FIG. 9 is a view showing an example of the imaging operation of the imaging apparatus according to an first modification;

FIG. 10 is a view showing a part of the configuration of an imaging apparatus according to a second modification;

FIG. 11 is a view showing a part of the configuration of an imaging apparatus according to a third modification;

FIG. 12 is a view showing a part of the configuration of an imaging apparatus according to a fourth modification; and

FIG. 13 is a view showing a part of the configuration of the imaging apparatus according to a fourth modification.

### DESCRIPTION OF THE EMBODIMENTS

An imaging apparatus according to an embodiment will now be described with reference to the accompanying drawings. Note that the embodiment is not limited to the following contents. In addition, the contents described in one embodiment or modification are similarly applied to another embodiment or modification in principle.

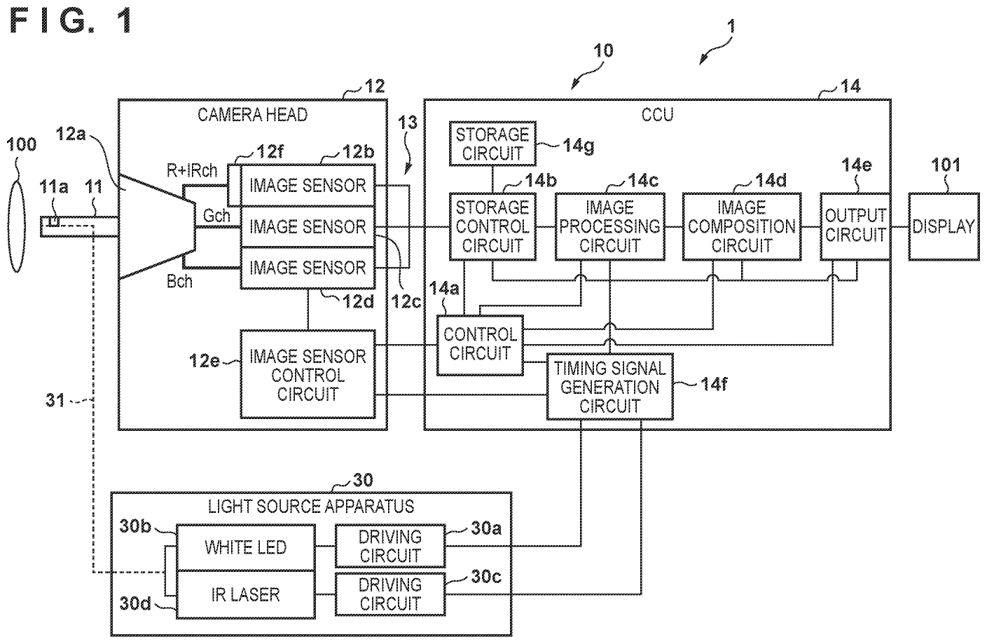

FIG. 1 is a block diagram showing an example of the configuration of an imaging system 1 including an imaging apparatus 10 according to this embodiment. As shown in FIG. 1, the imaging system 1 according to this embodiment includes the imaging apparatus 10, a light source apparatus 30, and an optical fiber 31.

The imaging apparatus 10 is used as, for example, a rigid endoscope for a medical application, which is an apparatus that captures the inside of a subject 100. The imaging apparatus 10 includes a scope 11, a camera head 12, a camera cable 13, and a CCU (Camera Control Unit) 14. Note that the imaging apparatus 10 is not limited only to the rigid endoscope.

The scope 11 is inserted into the inside of the subject 100 when performing imaging. An objective lens 11a is provided at the distal end of the scope 11.

The camera head 12 includes a prism 12a, a plurality of image sensors, and an image sensor control circuit 12e.

The prism 12a separates incident light into light components in three or more types of wavelength bands. For example, the prism 12a is a tricolor separating dichroic prism. For example, the prism 12a spectrally divides incident light into red (R+IR) light, green (G) light, and blue (B) light. The prism 12a is an example of an optical element.

The plurality of image sensors receive the light components in the three or more types of wavelength bands separated by the prism 12a, respectively. For example, the plurality of image sensors are CMOS (Complementary Metal Oxide Semiconductor) image sensors. For example, as the plurality of image sensors, image sensors 12b, 12c, and 12d receive the red (R+IR) light, the green (G) light, and the blue (B) light separated by the prism 12a, respectively. The image sensor 12b corresponds to, for example, red and infrared wavelength bands (expressed as “R+IRch (channel)” in FIG. 1), and is provided on the exit surface of the prism 12a for spectrally divided red light. The image sensor 12c corresponds to, for example, a green wavelength band (expressed as “Gch” in FIG. 1), and is provided on the exit surface of the prism 12a for spectrally divided green light. The image sensor 12d corresponds to, for example, a blue wavelength band (expressed as “Bch” in FIG. 1), and is provided on the exit surface of the prism 12a for spectrally divided blue light. The image sensors 12b, 12c, and 12d will sometime be referred to as the image sensor 12b on the R+IRch side, the image sensor 12b on the Gch side, and the image sensor 12b on the Bch side, respectively, hereinafter. The imaging surfaces of the image sensors 12b, 12c, and 12d are arranged to almost match the imaging surface of an optical system including the scope 11. The image sensors 12b, 12c, and 12d are examples of an imaging element.

Each of the image sensors 12b, 12c, and 12d includes a plurality of pixels (imaging pixels). The plurality of pixels are arranged in a matrix on the imaging surface. Under the driving control of the image sensor control circuit 12e, each pixel generates a video signal (electrical signal) by receiving light, and outputs the generated video signal. For example, each pixel of the image sensor 12b receives red light, thereby outputting an R signal (R video signal). In addition, each pixel of the image sensor 12c receives green light, thereby outputting a G signal (G video signal). Furthermore, each pixel of the image sensor 12d receives blue light, thereby outputting a B signal (b video signal). For example, the camera head 12 including the image sensors 12b, 12c, and 12d outputs an RGB signal to the CCU 14 via the camera cable 13. Note that an analog video signal is output from each of the image sensors 12b, 12c, and 12d. Alternatively, if each of the image sensors 12b, 12c, and 12d incorporates an A/D (Analog to Digital) converter (not shown), a digital video signal is output from each of the image sensors 12b, 12c, and 12d.

Here, the imaging apparatus 10 according to this embodiment is used when, for example, performing a surgical operation by ICG (IndoCyanine Green) fluorescence angiography for the subject 100. In this case, ICG is administered to the subject 100. ICG is excited by excitation light emitted by an IR laser 30d and emits near-infrared fluorescence (to be referred to as fluorescence hereinafter) of about 800 to 850 nm. In the ICG fluorescence angiography, a filter that cuts excitation light is provided between the scope 11 and the prism 12a, and the fluorescence is received by the image sensor 12b. That is, the image sensor 12b receives the fluorescence based on the excitation light, thereby outputting an R signal.

Each of the image sensors 12b, 12c, and 12d is a rolling shutter image sensor that repeats, for every frame (image), processing of sequentially starting exposure, at least on each row, from the first row to the final row of the plurality of pixels and outputting a video signal sequentially from a row that has undergone the exposure. Here, exposure means, for example, accumulating charges in the pixels.

The image sensor control circuit 12e drives and controls the image sensors 12b, 12c, and 12d based on a control signal output from a control circuit 14a to be described later and various kinds of synchronization signals output from a timing signal generation circuit 14f to be described later. For example, if the image sensors 12b, 12c, and 12d output analog video signals, the image sensor control circuit 12e appropriately applies a gain (analog gain) to each of the analog video signals output from the image sensors 12b, 12c, and 12d (amplifies the video signals) based on the control signal and the various kinds of synchronization signals, thereby controlling the image sensors 12b, 12c, and 12d such that the video signals multiplied by the gain are output to the CCU 14. Alternatively, if the image sensors 12b, 12c, and 12d output digital video signals, the image sensor control circuit 12e appropriately applies a gain (digital gain) to each of the digital video signals output from the image sensors 12b, 12c, and 12d based on the control signal and the various kinds of synchronization signals, thereby controlling the image sensors 12b, 12c, and 12d such that the video signals multiplied by the gain are output to the CCU 14.

The camera cable 13 is a cable that stores signal lines configured to transmit/receive video signals, control signals, and synchronization signals between the camera head 12 and the CCU 14.

The CCU 14 performs various kinds of image processing for a video signal output from the camera head 12 to generate image data to be displayed on a display 101, and outputs the image data to the display 101 connected to the CCU 14. Note that the video signal that has undergone the various kinds of image processing is image data representing an image to be displayed on the display 101.

The CCU 14 includes the control circuit 14a, a storage control circuit 14b, an image processing circuit 14c, an image composition circuit 14d, an output circuit 14e, the timing signal generation circuit 14f, and a storage circuit 14g. Note that when the image sensors 12b, 12c, and 12d output analog video signals, the CCU 14 includes an A/D converter and the like (not shown) as well. The A/D converter converts, for example, analog video signals output from the image sensors 12b, 12c, and 12d into digital video signals.

The control circuit 14a controls various kinds of constituent elements of the imaging apparatus 10. For example, the control circuit 14a outputs control signals to the image sensor control circuit 12e, the storage control circuit 14b, the image processing circuit 14c, the image composition circuit 14d, the output circuit 14e, and the timing signal generation circuit 14f, thereby controlling the circuits. The control circuit 14a loads the control program of the imaging apparatus 10, which is stored in the storage circuit 14g, and executes the loaded control program, thereby executing control processing of controlling the various kinds of constituent elements of the imaging apparatus 10. Alternatively, the control circuit 14a incorporates a storage circuit (not shown) and executes a control program stored in the storage circuit. The control circuit 14a is implemented by, for example, a processor such as an MPU (Micro-Processing Unit).

The storage control circuit 14b performs control of storing, in the storage circuit 14g, a video signal output from the camera head 12 based on a control signal output from the control circuit 14a and various kinds of synchronization signals output from the timing signal generation circuit 14f In addition, the storage control circuit 14b reads the video signal stored in the storage circuit 14g from each row based on the control signal and the synchronization signals. The storage control circuit 14b then outputs the read video signal of one row to the image processing circuit 14c.

The image processing circuit 14c performs various kinds of image processing for the video signal output from the storage control circuit 14b based on a control signal output from the control circuit 14a and various kinds of synchronization signals output from the timing signal generation circuit 14f The image processing circuit 14c thus generates image data representing an image to be displayed on the display 101. That is, the image processing circuit 14c generates the image based on the video signal. For example, the image processing circuit 14c applies a gain (digital gain) to the video signal output from the storage control circuit 14b, thereby adjusting the brightness of the image. The image processing circuit 14c may perform noise reduction processing of reducing noise or edge enhancement processing of enhancing edges for the video signal output from the storage control circuit 14b. The image processing circuit 14c outputs the video signal (image data representing the image to be displayed on the display 101) that has undergone the various kinds of image processing to the image composition circuit 14d.

The image composition circuit 14d composites video signals output from the image processing circuit 14c to generate composite image data based on a control signal output from the control circuit 14a and various kinds of synchronization signals output from the timing signal generation circuit 14f The image composition circuit 14d outputs the composite image data to the display 101.

For example, the storage control circuit 14b, the image processing circuit 14c, and the image composition circuit 14d are implemented by one processor such as a DSP (Digital Signal Processor). Alternatively, for example, the storage control circuit 14b, the image processing circuit 14c, the image composition circuit 14d, and the timing signal generation circuit 14f are implemented by one FPGA (Field Programmable Gate Array). Note that the control circuit 14a, the storage control circuit 14b, the image processing circuit 14c, and the image composition circuit 14d may be implemented by one processing circuit. The processing circuit is implemented by, for example, a processor.

The output circuit 14e outputs the composite image data output from the image composition circuit 14d to the display 101. The display 101 thus displays a composite image represented by the composite image data. The composite image is an example of an image. The output circuit 14e is implemented by, for example, an HDMI® (High-Definition Multimedia Interface) driver IC (Integrated Circuit), an SDI (Serial Digital Interface) driver IC, or the like.

The timing signal generation circuit 14f unitarily manages various kinds of timings such as the emission timing of light from the light source apparatus 30, the exposure timings and video signal output timings of the image sensors 12b, 12c, and 12d, and the control timing of the storage circuit 14g by the storage control circuit 14b.

The timing signal generation circuit 14f generates various kinds of synchronization signals such as a horizontal synchronization signal and a vertical synchronization signal, and other synchronization signals used to synchronize the entire imaging apparatus 10 based on a clock signal generated by an oscillation circuit (not shown). The timing signal generation circuit 14f outputs the generated various kinds of synchronization signals to the image sensor control circuit 12e, the control circuit 14a, the storage control circuit 14b, the image processing circuit 14c, the image composition circuit 14d, and the output circuit 14e.

In addition, the timing signal generation circuit 14f generates a light source control signal based on the clock signal and a control signal output from the control circuit 14a. The light source control signal is a control signal used to control light emitted from the light source apparatus 30 and also synchronize the entire imaging system 1. The timing signal generation circuit 14f outputs the generated light source control signal to the light source apparatus 30.

For example, the light source control signal has a rectangular waveform, and takes two levels (states), that is, high level and low level. For example, the light source control signal is a control signal that causes the light source apparatus 30 to emit light during high level, and stops emission of light from the light source apparatus 30 during low level.

The storage circuit 14g is implemented by, for example, a RAM (Random Access Memory), a ROM (Read Only Memory), a semiconductor memory element such as a flash memory, a hard disk, an optical disk, or the like. The ROM (or flash memory or hard disk) stores various kinds of programs. For example, the ROM stores a control program to be executed by the control circuit 14a. In addition, video signals are temporarily stored in the RAM by the storage control circuit 14b.

The light source apparatus 30 emits white light or excitation light based on the light source control signal. The light source apparatus 30 includes a driving circuit 30a, a white LED (Light Emitting Diode) 30b, a driving circuit 30c, and an IR laser 30d.

The driving circuit 30a performs driving control of driving and turning on the white LED 30b based on the light source control signal output from the timing signal generation circuit 14f The white LED 30b emits white light under the driving control of the driving circuit 30a. The white light is, for example, visible light.

The driving circuit 30c performs driving control of driving the IR laser 30d and causing the IR laser 30d to emit excitation light based on the light source control signal output from the timing signal generation circuit 14f. The IR laser 30d emits excitation light under the driving control of the driving circuit 30c. Note that fluorescence (fluorescence based on the excitation light) emitted from the ICG excited by the excitation light is received by the image sensor 12b.

The optical fiber 31 guides the white light and the excitation light from the light source apparatus 30 to the distal end portion of the scope 11 and outputs the light from the distal end portion of the scope 11.

Here, as shown in FIG. 1, the camera head 12 further includes a filter 12f.

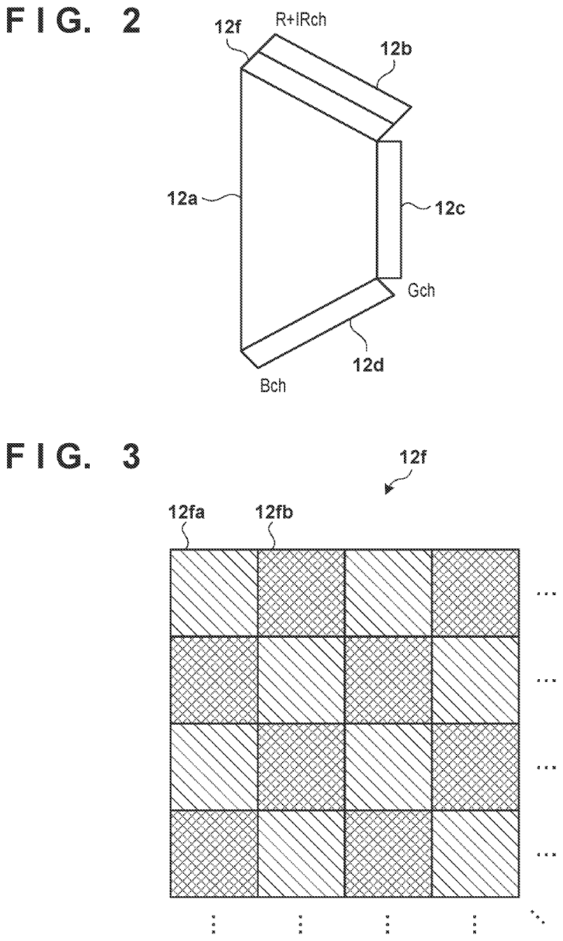

FIG. 2 is a view showing a part of the configuration of the imaging apparatus 10 according to this embodiment. For example, the filter 12f is provided between the exit surface (R+IRch) of the prism 12a for spectrally divided red light and the imaging surface of the image sensor 12b. The filter 12f further separates the wavelength band of light that has entered image sensor 12b into two or more types of wavelength bands. For example, the filter 12f further separates the wavelength band of light that has entered image sensor 12b into a red wavelength band and an infrared wavelength band.

FIG. 3 is a view showing an example of the filter 12f according to this embodiment. The filter 12f includes first filters 12fa and second filters 12fb. For example, the filter 12f is a filter having a checkered pattern, and the first filters 12fa and the second filters 12fb are alternately arranged. More specifically, the filter 12f is arranged on the imaging plane side of the image sensor 12b such that one of the first filters 12fa and the second filters 12fb faces each pixel. For example, the first filters 12fa pass light in the red wavelength band that is visible light. The second filters 12fb pass light in the infrared wavelength band.

An example of the configuration of the imaging apparatus 10 of the imaging system 1 according to this embodiment has been described above. An imaging apparatus according to a comparative example will be described here. The imaging apparatus according to the comparative example is, for example, the imaging apparatus 10 shown in FIG. 1, which does not include the filter 12f.

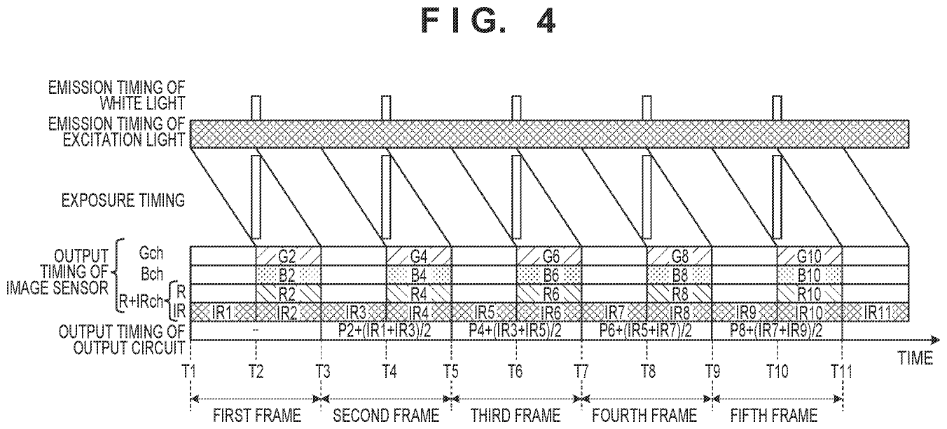

FIG. 4 is a view showing an example of the imaging operation of the imaging apparatus according to the comparative example. FIG. 4 shows an example of the relationship between the emission timings of white light and excitation light emitted from the light source apparatus 30, the exposure timings of the rows of the plurality of pixels provided in the image sensors 12b, 12c, and 12d of the imaging apparatus 10, the output timings of video signals output from the image sensors 12b, 12c, and 12d, and the output timings of a video signal output from the output circuit 14e. In FIG. 4, the abscissa represents time. In the comparative example, the frame rate of a video signal (image) output from the imaging apparatus 10 to the display 101 is 60 [fps (frame per second)], and the read period is 1/120 [s]. That is, the period of outputting a video signal of one frame from the imaging apparatus 10 to the display 101 is 1/60 [s], and the read period are 1/120 [s].

In the comparative example, in each frame, time-divisional control is performed in which an R signal “IR” is acquired during the first read period of 1/120 [s], and an RGB signal is acquired during the second read period of 1/120 [s]. The imaging operation of the imaging apparatus according to the comparative example will be described below in detail.

First, at the start of imaging, the control circuit 14a outputs a control signal to the timing signal generation circuit 14f to cause it to output a first light source control signal that causes the IR laser 30d to continuously emit excitation light. The timing signal generation circuit 14f outputs the first light source control signal to the driving circuit 30c based on the control signal, and the driving circuit 30c drives the IR laser 30d based on the first light source control signal, thereby causing the IR laser 30d to continuously emit excitation light.

Also, in each frame, only during a blanking period that is a period when the first read period switches to the second read period, the control circuit 14a outputs a control signal to the timing signal generation circuit 14f to cause it to output a second light source control signal that causes the white LED 30b to emit white light. The timing signal generation circuit 14f outputs the second light source control signal to the driving circuit 30a based on the control signal, and the driving circuit 30a drives the white LED 30b based on the second light source control signal, thereby causing the white LED 30b to emit white light.

For example, in the first frame, during the first read period of 1/120 [s] from time T1 to time T2, a video signal (an R signal “IR1” to be described later) is output from the image sensor 12b. More specifically, the control circuit 14a outputs a control signal to the image sensor control circuit 12e to cause the image sensor 12b to output a video signal during the first read period of 1/120 [s]. The image sensor control circuit 12e drives and controls the image sensor 12b based on the control signal. As a result, during the read period of 1/120 [s] from time T2 to time T2, the image sensor 12b receives light in the infrared wavelength band, which has exited from the prism 12a, and outputs video signals from all rows as the R signal “IR1”. The storage control circuit 14b temporarily stores, in the storage circuit 14g, the video signal (R signal “IR1”) output from each row of the image sensor 12b.

Additionally, in the first frame, during the first read period of 1/120 [s] from time T1 to time T2, exposure is sequentially started on each row from the first row to the final row of the plurality of pixels of each of the image sensors 12b, 12c, and 12d. Here, a time difference corresponding to the read period is present between the exposure start and the exposure end (output start). For example, in the first row, exposure is performed during the period from the time T1 to the time T2, and output is performed at the time T2. In the second row, exposure is performed during the period from the time T2 to time T3, and output is performed at the time T3. More specifically, the control circuit 14a outputs a control signal to the image sensor control circuit 12e to cause the image sensors 12b, 12c, and 12d to output video signals during the second read period of 1/120 [s]. The image sensor control circuit 12e drives and controls the image sensors 12b, 12c, and 12d based on the control signal. As a result, the image sensor 12b receives light in the red and infrared wavelength bands, which has exited from the prism 12a, and outputs video signals from all rows as an R signal “R2+IR2” during the second read period of 1/120 [s] from the time T2 to the time T3. The image sensor 12c receives light in the green wavelength band, which has exited from the prism 12a, and outputs video signals from all rows as a G signal “G2”. The image sensor 12d receives light in the blue wavelength band, which has exited from the prism 12a, and outputs video signals from all rows as a B signal “B2”. The storage control circuit 14b temporarily stores, in the storage circuit 14g, an RGB signal “P2” as the video signals output from the rows of the image sensors 12b, 12c, and 12d. The RGB signal “P2” represents the composite signal of the R signal “R2+IR2”, the G signal “G2”, and the B signal “B2”. That is, the RGB signal “P2” includes the R signal “R2+IR2” output from the image sensor 12b that has received the white light and the fluorescence based on the excitation light, the G signal “G2” output from the image sensor 12c that has received the white light, and the B signal “B2” output from the image sensor 12d that has received the white light. In other words, the RGB signal “P2” shown in FIG. 4 as the comparative example is a signal “W2+IR2” including the signal “W2=R2+G2+B2” based on the white light and the signal “IR2” based on the fluorescence.

Next, in the second frame, the image sensor 12b outputs a video signal as an R signal “IR3” during the first read period of 1/120 [s] from the time T3 to time T4, and the storage control circuit 14b temporarily stores, in the storage circuit 14g, the video signal (R signal “IR3”) output from the image sensor 12b. Also, the image sensors 12b, 12c, and 12d output video signals as an R signal “R4+IR4”, a G signal “G4”, and a B signal “B4”, respectively, during the second read period of 1/120 [s] from the time T4 to time T5. The storage control circuit 14b temporarily stores, in the storage circuit 14g, an RGB signal “P4” as the video signals output from the image sensors 12b, 12c, and 12d. The RGB signal “P4” represents the composite signal of the R signal “R4+IR4”, the G signal “G4”, and the B signal “B4”.

Here, in the second frame, for example, the video signal of the first frame stored in the storage circuit 14g is output from the output circuit 14e to the display 101 via the image processing circuit 14c and the image composition circuit 14d. More specifically, the image processing circuit 14c generates a first display image based on the RGB signal “P2”. Next, the image composition circuit 14d composites, for example, the R signal “IR1” and the R signal “IR3”, thereby generating a composite image “(IR1+IR3)/2”. Next, the image composition circuit 14d extracts, as a target, a portion with a brightness equal to or larger than a threshold from the generated composite image, and generates a fluorescent image that is a marker formed by adding a fluorescent color to the extracted portion. The fluorescent color is a color assigned to represent fluorescence when the marker (fluorescent image) is generated, and shows, for example, green of high saturation. The image composition circuit 14d superimposes the generated fluorescent image on the first display image generated by the image processing circuit 14c, thereby generating a second display image. The second display image generated by the image composition circuit 14d is output from the output circuit 14e to the display 101 during the period of 1/60 [s]. From the third frame as well, processing similar to the above-described processing is performed.

As described above, in the comparative example, in each frame, time-divisional control is performed in which an R signal “IR” is acquired during the first read period of 1/120 [s], and an RGB signal is acquired during the second read period of 1/120 [s]. In the time-divisional control, the light source apparatus 30 is caused to emit white light during a blanking period that is a very short period from the end of the first read period to the start of the second read period of each frame. In other words, the white light is emitted only during the blanking period. Hence, in the comparative example, by time-divisional control, the image sensors 12b, 12c, and 12d receive only light during a very short time corresponding to the blanking period. For this reason, if brightness at the time of imaging is not sufficient, sensitivity lowers. For example, sensitivity concerning white light lowers twice or more as compared to a case in which the image sensors 12b, 12c, and 12d receive light during a time corresponding to 1/120 [s]. Hence, for a user such as a doctor who observes images, image quality may not be sufficient for observation.

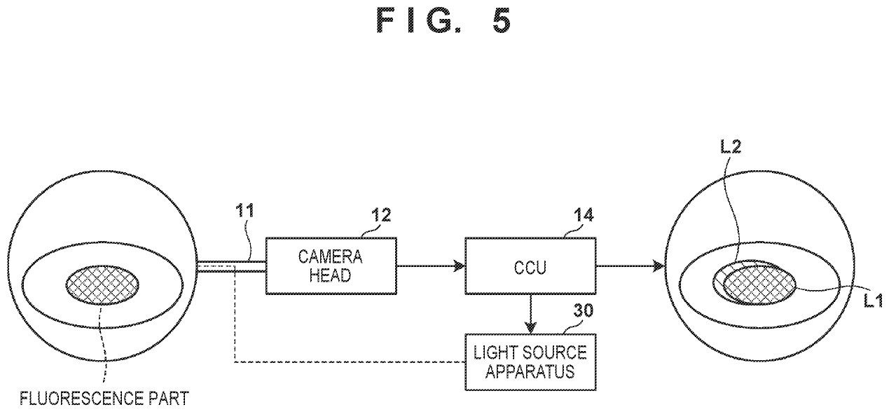

In the comparative example, time-divisional control is performed. Hence, in a case where a target is moving, when a marker (fluorescent image) is generated based on the R signal “IR”, the position of the target indicated by a fluorescent color (green of high saturation) and the actual position of the target may have a deviation. For example, if the target is not moving, since no deviation occurs between the position of the target indicated by green of high saturation and the actual position of the target, the user does not feel uncomfortable. However, if the target is moving, a deviation occurs between a position L1 of the target indicated by green of high saturation and an actual position L2 of the target, as shown in FIG. 5, because of time-divisional control, and the user feels uncomfortable. Here, the position L1 shown in FIG. 5 is a position when the R signal “IR” is acquired, and the position L2 shown in FIG. 5 is a position when an RGB signal is acquired. The RGB signal includes an R signal “R+IR” output from the image sensor 12b that has received white light and fluorescence based on excitation light. For this reason, when a display image is generated, the target tends to appear redder than it should be. Hence, if a deviation occurs between the position L1 of the target indicated by green of high saturation and the actual position L2 of the target that appears redder than it should be, the user feels uncomfortable. As described above, in the comparative example, time-divisional control is performed. For this reason, if the target is moving, the effective imaging range when imaging the target becomes narrow.

Also, in the comparative example, if the frame rate of the video signal (image) output from the imaging apparatus 10 to the display 101 is 60 [fps], since the image sensors 12b, 12c, and 12d are driven and controlled in a time of 1/120 [s] by performing the above-described time-divisional control, power consumption increases. Additionally, in the comparative example, if the frame rate is 60 [fps], since the R signal “IR” and the RGB signal are acquired in two frames at an interval of 1/60 [s] by time-divisional control, the number of signal processing operations increases. Hence, if the diameter per cable such as the camera cable 13 or the number of cables is increased because of the increase in the number of signal processing operations, the diameter of the entire cable becomes large.

In addition, the imaging apparatus according to the comparative example includes the prism 12a that is a tricolor separating dichroic prism, and performs the above-described time-divisional control, thereby separating incident light into four color light components, that is, light in the red wavelength band, light in the infrared wavelength band, light in the green wavelength band, and light in the blue wavelength band. As a method of performing four color separation without using the method as in the comparative example, a single sensor with a four-color separation filter is used, or a four-color separation prism is used. However, if the single sensor with the four-color separation filter is used, each channel can use only ¼ of all pixels, and therefore, sensitivity or resolution lowers. If the four-color separation prism is used, the cost is higher than in a case where a tricolor separating dichroic prism is used, and the camera head 12 that is an imaging portion becomes bulky.

The imaging apparatus 10 according to this embodiment performs the following processing to ensure image quality sufficient for the user to observe. The imaging apparatus 10 according to this embodiment includes the prism 12a, and the plurality of image sensors. The prism 12a is an optical element that separates incident light into light components in three or more types of wavelength bands. The plurality of image sensors are imaging elements that receive the light components in three or more types of wavelength bands separated by the prism 12a, respectively. At least one image sensor of the plurality of image sensors has a function of further separating the wavelength band of light that has entered the image sensor into two or more types of wavelength bands. More specifically, the prism 12a separates incident light into light in the red and infrared wavelength bands, light in the green wavelength band, and light in the blue wavelength band. Of the plurality of image sensors 12b, 12c, and 12d, the image sensor 12b includes the filter 12f that further separates the wavelength band of light that has entered the image sensor 12b into the red wavelength band and the infrared wavelength band. For example, the filter 12f includes the first filters 12fa that pass visible light, and the second filters 12fb that pass light in the infrared wavelength band.

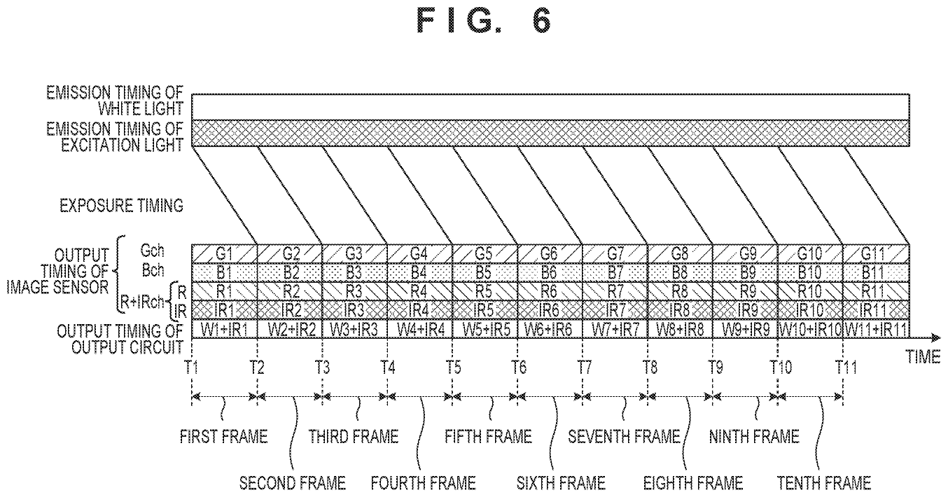

FIG. 6 is a view showing an example of the imaging operation of the imaging apparatus 10 according to this embodiment. FIG. 6 shows an example of the relationship between the emission timings of white light and excitation light emitted from the light source apparatus 30, the exposure timings of the rows of the plurality of pixels provided in the image sensors 12b, 12c, and 12d of the imaging apparatus 10, the output timings of video signals output from the image sensors 12b, 12c, and 12d, and the output timings of a video signal output from the output circuit 14e. In FIG. 6, the abscissa represents time. In this embodiment, the frame rate of a video signal (image) output from the imaging apparatus 10 to the display 101 is 120 [fps], and the read period is 1/120 [s]. That is, the period of outputting a video signal of one frame from the imaging apparatus 10 to the display 101 and the read period are 1/120 [s].

First, at the start of imaging, the control circuit 14a outputs a control signal to the timing signal generation circuit 14f to cause it to output a first light source control signal that causes the IR laser 30d to continuously emit excitation light. The timing signal generation circuit 14f outputs the first light source control signal to the driving circuit 30c based on the control signal, and the driving circuit 30c drives the IR laser 30d based on the first light source control signal, thereby causing the IR laser 30d to continuously emit excitation light.

Also, at the start of imaging, the control circuit 14a outputs a control signal to the timing signal generation circuit 14f to cause it to output a second light source control signal that causes the white LED 30b to continuously emit white light. The timing signal generation circuit 14f outputs the second light source control signal to the driving circuit 30a based on the control signal, and the driving circuit 30a drives the white LED 30b based on the second light source control signal, thereby causing the white LED 30b to continuously emit white light.

For example, in the first frame, during the read period of 1/120 [s] from time T1 to time T2, exposure is sequentially started on each row from the first row to the final row of the plurality of pixels of each of the image sensors 12b, 12c, and 12d. More specifically, the control circuit 14a outputs a control signal to the image sensor control circuit 12e to cause the image sensors 12b, 12c, and 12d to output video signals during the read period of 1/120 [s]. The image sensor control circuit 12e drives and controls the image sensors 12b, 12c, and 12d based on the control signal. As a result, the image sensor 12b receives light in the red and infrared wavelength bands, which has exited from the prism 12a, and outputs video signals from all rows as R signals “R1” and “IR1” during the read period of 1/120 [s]. More specifically, of the light in the red and infrared wavelength bands, which has exited from the prism 12a, the image sensor 12b receives light in the red wavelength band that has passed through the first filters 12fa of the filter 12f, and outputs the R signal “R1”. In addition, of the light in the red and infrared wavelength bands, which has exited from the prism 12a, the image sensor 12b receives light in the infrared wavelength band that has passed through the second filters 12fb of the filter 12f, and outputs the R signal “IR1”. The image sensor 12c receives light in the green wavelength band that has exited from the prism 12a, and outputs video signals from all rows as a G signal “G1”. The image sensor 12d receives light in the blue wavelength band that has exited from the prism 12a, and outputs video signals from all rows as a B signal “B1”. In this case, an RGB signal “W1” and the R signal “IR1” are output as video signals from the image sensors 12b, 12c, and 12d. The RGB signal “W1” represents the composite signal of the R signal “R1”, the G signal “G1”, and the B signal “B1”. That is, the RGB signal “W1” includes an R signal “R2” output from the image sensor 12b that has received white light via the first filters 12fa, a G signal “G2” output from the image sensor 12c that has received white light, and a B signal “B2” output from the image sensor 12d that has received white light.

Next, in the second frame, the image sensors 12b, 12c, and 12d output video signals as R signals “R2” and “IR2”, a G signal “G2”, and a B signal “B2”, respectively, during a read period of 1/120 [s] from the time T2 to time T3. In this case, an RGB signal “W2” and the R signal “IR2” are output as video signals from the image sensors 12b, 12c, and 12d. The RGB signal “W2” represents the composite signal of the R signal “R2”, the G signal “G2”, and the B signal “B2”.

Here, the video signals output from the image sensors 12b, 12c, and 12d are changed to the display image of the first frame via the image processing circuit 14c and the image composition circuit 14d, and quickly output from the output circuit 14e to the display 101. More specifically, the image processing circuit 14c generates a first display image based on the RGB signal “W1”. Next, for example, the image composition circuit 14d extracts, as a target, a portion with a brightness equal to or larger than a threshold from the image represented by the R signal “IR1”, and generates a fluorescent image that is a marker formed by adding a fluorescent color to the extracted portion. The fluorescent color is a color assigned to represent fluorescence when the marker (fluorescent image) is generated, and shows, for example, green of high saturation. The image composition circuit 14d superimposes the generated fluorescent image on the first display image generated by the image processing circuit 14c, thereby generating a second display image. The second display image generated by the image composition circuit 14d is output from the output circuit 14e to the display 101 during the period of 1/120 [s]. From the second frame as well, processing similar to the above-described processing is performed.

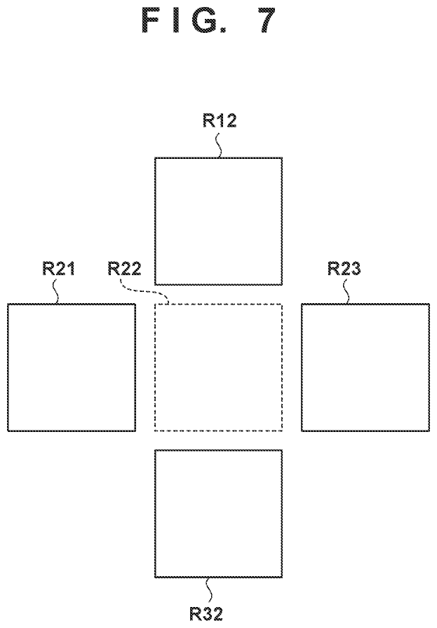

Here, concerning the R signals “R” and “IR”, since the spatial resolution (resolution) is halved by the configuration of the filter 12f, the image processing circuit 14c performs pixel interpolation processing before the above-described display image is generated. For example, because of the configuration of the filter 12f, since pixels that output the R signal “R” and pixels that output the R signal “IR” are alternately arranged in the image sensor 12b, the pixels need to be interpolated. As shown in FIG. 7, if four pixels that output the R signal “R” are pixels R12, R21, R23, and R32, the image processing circuit 14c performs processing of interpolating a pixel R22 by calculating the average of the pixel values of the pixels R12, R21, R23, and R32. Since scattering of a red component is generally large, there is no problem if the resolution of an image obtained from the R signal “R” is not so high when the above-described pixel interpolation processing is performed. The image processing circuit 14c performs the pixel interpolation processing similarly for the R signal “IR” as well. After the processing, the image processing circuit 14c generates the above-described first display image. The image composition circuit 14d generates the above-described fluorescent image, and superimposes the fluorescent image on the first display image, thereby generating a second display image. At this time, the second display image is output from the output circuit 14e to the display 101 during the period of 1/120 [s].

As described above, since the imaging apparatus 10 according to this embodiment includes the filter 12f that further separates the wavelength band of light that has entered the image sensor 12b into the red wavelength band and the infrared wavelength band, time-divisional control as in the comparative example need not be performed. In this embodiment, since the light source apparatus 30 is caused to emit white light in each frame, the exposure period of the image sensors 12b, 12c, and 12d is the same as the period of outputting a video signal of one frame from the imaging apparatus 10 to the display 101. That is, in this embodiment, since the image sensors 12b, 12c, and 12d receive light during the same period as the period of outputting a video signal of one frame from the imaging apparatus 10 to the display 101, brightness of a target or background can sufficiently be ensured, and high sensitivity and high resolution are implemented. Hence, in this embodiment, it is possible to ensure image quality sufficient for the user to observe.

In this embodiment, for example, if the frame rate of a video signal (image) output from the imaging apparatus 10 to the display 101 is 60 [fps], the image sensors 12b, 12c, and 12d are driven and controlled in a time of 1/60 [s]. For this reason, in this embodiment, power consumption can be suppressed as compared to the comparative example in which the image sensors 12b, 12c, and 12d are driven and controlled in a time of 1/120 [s] in a case where the frame rate is 60 [fps]. Additionally, in this embodiment, since time-divisional control as in the comparative example need not be performed, the R signal “IR” and the RGB signal can be acquired at an interval of 1/60 [s] in a case where the frame rate is 60 [fps]. For this reason, in this embodiment, the number of signal processing can be reduced as compared to the comparative example in which the R signal “IR” and the RGB signal are acquired in at an interval of 1/60 [s] by time-divisional control in a case where the frame rate is 60 [fps]. In this embodiment, when decreasing the diameter per cable such as the camera cable 13 or the number of cables along with the decrease in the number of signal processing operations, the diameter of the entire cable can be made smaller than in the comparative example.

In addition, the imaging apparatus 10 according to this embodiment includes the prism 12a that is tricolor separating dichroic prism, and the filter 12f that is a color filter, thereby separating incident light into four color light components, that is, light in the red wavelength band, light in the infrared wavelength band, light in the green wavelength band, and light in the blue wavelength band. Hence, in this embodiment, high sensitivity and high resolution are implemented as compared to a case in which a single sensor with a four-color separation filter is used. For example, in the single sensor with the four-color separation filter, each channel can use only ¼ of all pixels. In this embodiment, however, Gch and Bch can use all pixels. In particular, since Gch greatly affects the resolution, the configuration of this embodiment is effective from the viewpoint of high sensitivity and high resolution as well. The configuration of this embodiment is relatively inexpensive as compared to a case in which a four-color separation prism is used, and implements size reduction of the camera head 12 that is an imaging portion.

(First Modification)

Note that in this embodiment, in each frame, the light source apparatus 30 causes the white LED 30b to continuously emit white light, and causes the IR laser 30d to continuously emit excitation light. However, the present invention is not limited to this.

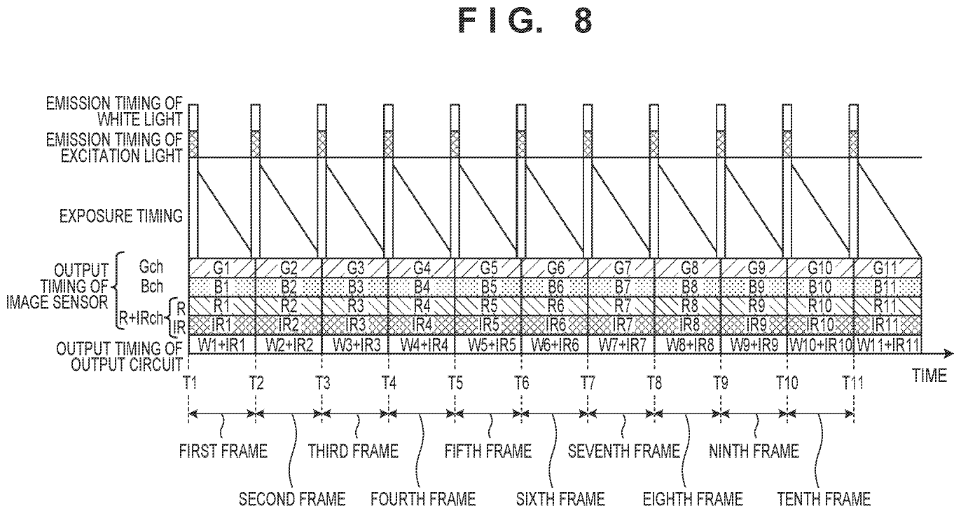

As the first modification, for example, if the brightness in imaging is sufficient, as shown in FIG. 8, in each frame, only during the blanking period, the light source apparatus 30 may cause the white LED 30b to emit white light, and may cause the IR laser 30d to emit excitation light.

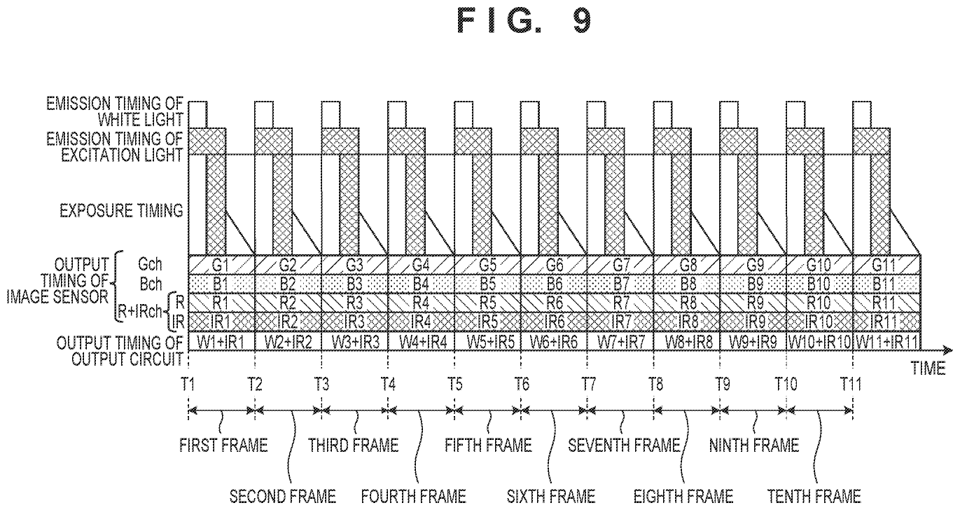

If the brightness in imaging is more satisfactory than in the example shown in FIG. 6 but lower than in the example shown in FIG. 8, for example, in each frame, the light source apparatus 30 may cause the white LED 30b to emit white light, and may cause the IR laser 30d to emit excitation light during a time as shown in FIG. 9. More specifically, in the example shown in FIG. 9, if the brightness is insufficient only by emitting white light during the blanking period, in each frame, the light source apparatus 30 causes the white LED 30b to emit white light during a first time longer than the blanking period. If the brightness is insufficient only by emitting excitation light during the first time, in each frame, the light source apparatus 30 causes the IR laser 30d to emit excitation light during a second time longer than the first time. In this case, the excitation light is emitted at the same time as the white light and also emitted longer than the white light. That is, the example shown in FIG. 9 shows a case in which the exposure period of white light and excitation light is set longer than the blanking period, and a case in which the exposure period of excitation light is set longer than the exposure period of white light by adjusting the brightness.

The imaging apparatus 10 according to the first modification need not perform time-divisional control as in the comparative example and implements high sensitivity and high resolution even in the example shown in FIG. 8 and the example shown in FIG. 9, as in the example shown in FIG. 6. Hence, in the first modification, it is possible to ensure image quality sufficient for the user to observe.

(Second Modification)

Also, in this embodiment, as the second modification, the resolution of an image may be raised using a method called half pixel shift.

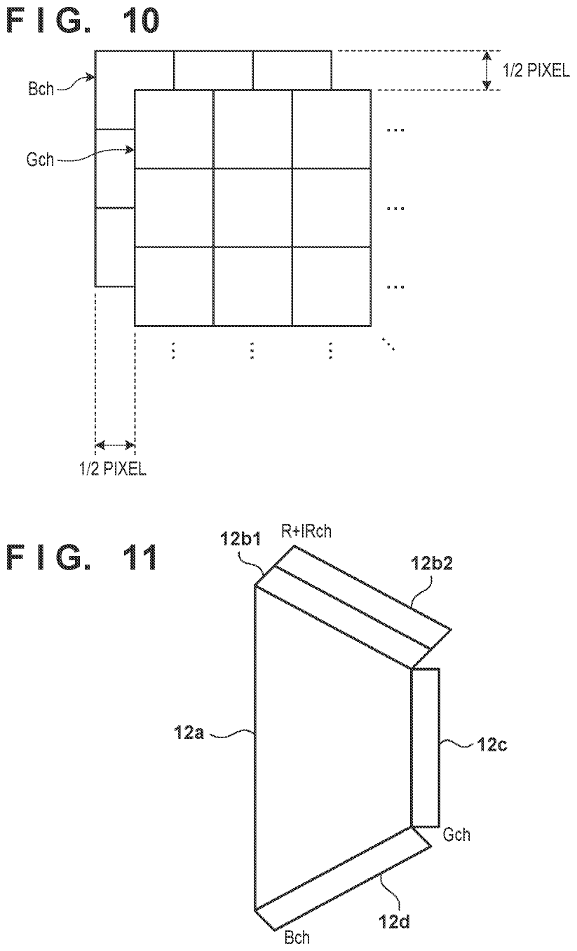

For example, the pixels of the image sensor 12c (the image sensor 12c on the Gch side) corresponding to the green wavelength band in the image sensors 12b, 12c, and 12d are arranged with a shift of a half pixel in at least one direction of the horizontal direction and/or the vertical direction with respect to the pixels of the image sensor 12d (the image sensor 12d on the Bch side) corresponding to the blue wavelength band. In the example shown in FIG. 10, the pixels of the image sensor 12c on the Gch side are arranged with a shift of a half pixel in the horizontal direction and the vertical direction with respect to the pixels of the image sensor 12d on the Bch side. Hence, in the second modification, the resolution of an image can be doubled.

(Third Modification)

The imaging apparatus 10 according to this embodiment includes the prism 12a that is a tricolor separating dichroic prism, and the filter 12f that is a color filter, thereby separating incident light into four color light components, that is, light in the red wavelength band, light in the infrared wavelength band, light in the green wavelength band, and light in the blue wavelength band. However, the present invention is not limited to this.

As the third modification, for example, the imaging apparatus 10 may include a stacked image sensor, instead of including the filter 12f. As shown in FIG. 11, the image sensor 12b includes stacked image sensors 12b1 and 12b2, and further separates the wavelength band of light that has entered the image sensor 12b into the red wavelength band and the infrared wavelength band. For example, the image sensor 12b1 is provided on the exit surface of the prism 12a for spectrally divided red light, and receives light in the red wavelength band of the light in the red and infrared wavelength bands, which has exited from the prism 12a, and outputs the R signal “R”. That is, the R signal “R” represents a signal output from the image sensor 12b1 that has received white light. The image sensor 12b2 is provided on the exit surface of the image sensor 12b1, and receives light in the infrared wavelength band of the light in the red and infrared wavelength bands, which has exited from the prism 12a, and outputs the R signal “IR”. That is, the R signal “IR” represents a signal output from the image sensor 12b2 that has received fluorescence based on excitation light.

In the third modification, the above-described stacked image sensors 12b1 and 12b2 are provided, thereby obviating the necessity of performing time-divisional control as in the comparative example and implementing high sensitivity and high resolution, as in a case where the filter 12f is provided. Hence, in the third modification, it is possible to ensure image quality sufficient for the user to observe.

(Fourth Modification)

In the imaging apparatus 10 according to this embodiment, the prism 12a separates incident light into light components in two or more types of wavelength bands, and at least one image sensor of the plurality of image sensors has a function of further separating the wavelength band of the incident light into two or more types of wavelength bands. At least one of the two or more types of wavelength bands is the infrared wavelength band. For example, the prism 12a separates incident light into light in the red and infrared wavelength bands, and at least one of light in the green wavelength band and/or light in the blue wavelength band. More specifically, as shown in FIG. 1, the prism 12a separates incident light into light in the red and infrared wavelength bands, light in the green wavelength band, and light in the blue wavelength band, and the image sensor 12b of the image sensors 12b, 12c, and 12d that are the plurality of image sensors includes the filter 12f that further separates the wavelength band of light that has entered the image sensor 12b into the red wavelength band and the infrared wavelength band. However, the present invention is not limited to the above-described embodiment.

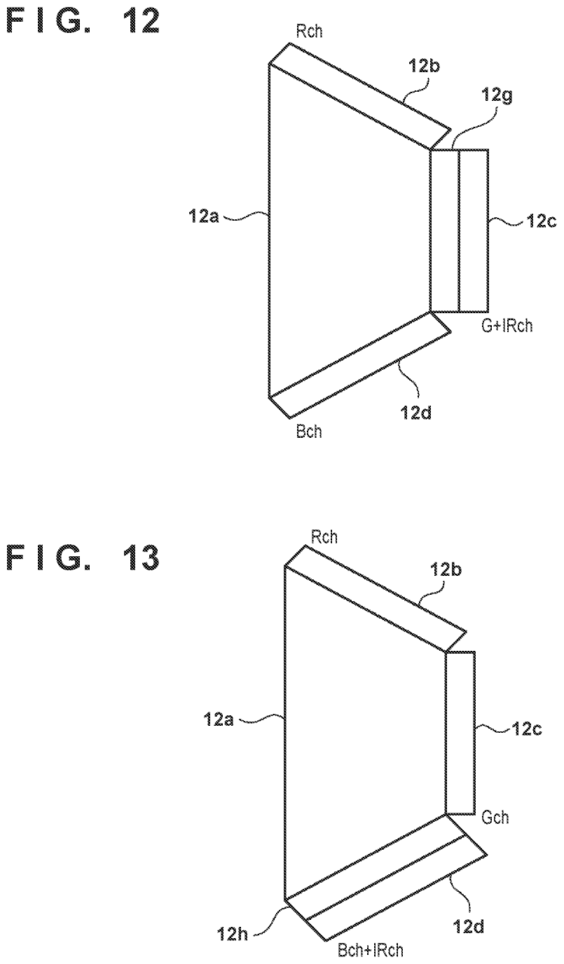

As the fourth modification, as shown in FIG. 12, the prism 12a separates incident light into light in the red wavelength band, light in the green and infrared wavelength bands, and light in the blue wavelength band. Of the image sensors 12b, 12c, and 12d, the image sensor 12c includes a filter 12g that further separates the wavelength band of light that has entered the image sensor 12c into the green wavelength band and the infrared wavelength band. The filter 12g is a filter having a checkered pattern, like the filter 12f, in which first filters that pass light in the green wavelength band that is visible light and second filters that pass light in the infrared wavelength band are alternately arranged.

Alternatively, as shown in FIG. 13, the prism 12a separates incident light into light in the red wavelength band, light in the green wavelength band, and light in the blue and infrared wavelength bands. Of the image sensors 12b, 12c, and 12d, the image sensor 12d includes a filter 12h that further separates the wavelength band of light that has entered the image sensor 12d into the blue wavelength band and the infrared wavelength band. The filter 12h is a filter having a checkered pattern, like the filter 12f, in which first filters that pass light in the blue wavelength band that is visible light and second filters that pass light in the infrared wavelength band are alternately arranged.

In the fourth modification, the filter 12g or the filter 12h is provided, thereby obviating the necessity of performing time-divisional control as in the comparative example and implementing high sensitivity and high resolution, as in a case where the filter 12f is provided. Hence, in the fourth modification, it is possible to ensure image quality sufficient for the user to observe. In addition, since the green or blue wavelength band is far apart from the infrared wavelength band, as compared to the red wavelength band, there is an advantage that the green or blue wavelength band and the infrared wavelength band can easily be separated in the configuration according to the fourth modification.

Note that the combinations for separating incident light are not limited to the above-described contents, and the following combinations can also be considered.

For example, the prism 12a may separate incident light into light in the green, red, and infrared wavelength bands and light in the blue wavelength band, and the image sensor 12b may further separate, by a filter, the wavelength band of the incident light into the green wavelength band, the red wavelength band, and the infrared wavelength band. The prism 12a may separate incident light into light in the blue, red, and infrared wavelength bands and light in the green wavelength band, and the image sensor 12b may further separate, by a filter, the wavelength band of the incident light into the blue wavelength band, the red wavelength band, and the infrared wavelength band. The prism 12a may separate incident light into light in the red and infrared wavelength bands and light in the green and blue wavelength bands, the image sensor 12b may further separate the wavelength band of the incident light into the red wavelength band and the infrared wavelength band, and one of the image sensors 12c and 12d may further separate, by a filter, the wavelength band of the incident light into the green wavelength band and the blue wavelength band. The prism 12a may separate incident light into light in the green and infrared wavelength bands and light in the blue and red wavelength bands, the image sensor 12c may further separate, by a filter, the wavelength band of the incident light into the green wavelength band and the infrared wavelength band, and one of the image sensors 12b and 12d may further separate, by a filter, the wavelength band of the incident light into the blue wavelength band and the red wavelength band. The prism 12a may separate incident light into light in the blue and infrared wavelength bands and light in the green and red wavelength bands, the image sensor 12d may further separate the wavelength band of the incident light into the blue wavelength band and the infrared wavelength band, and one of the image sensors 12b and 12c may further separate, by a filter, the wavelength band of the incident light into the green wavelength band and the red wavelength band.

Additionally, in the above-described embodiment, if the prism 12a that separates incident light into light in the red and infrared wavelength bands, light in the green wavelength band, and light in the blue wavelength band is used, the image sensor 12b on the R+IRch side may include not the filter 12f shown in FIGS. 1 to 3 but a filter in which first filters that are mere transmission filters and second filters that pass light in the infrared wavelength band are alternately arranged. In this case, the first filters that are transmission filters pass light in the red and infrared wavelength bands. There is an advantage that the filter having a checkered pattern in which the transmission filters are arranged can easily be manufactured at a low manufacturing cost, as compared to the filter 12f.

Similarly, in the fourth modification, if the prism 12a that separates incident light into light in the red wavelength band, light in the green and infrared wavelength bands, and light in the blue wavelength band is used, the image sensor 12c on the G+IRch side may include not the filter 12g shown in FIG. 12 but a filter in which first filters that are transmission filters and second filters that pass light in the infrared wavelength band are alternately arranged, as described above. In this case, the first filters that are transmission filters pass light in the green and infrared wavelength bands. Also, if the prism 12a that separates incident light into light in the red wavelength band, light in the green wavelength band, and light in the blue and infrared wavelength bands is used, the image sensor 12d on the B+IRch side may include not the filter 12h shown in FIG. 13 but a filter in which first filters that are transmission filters and second filters that pass light in the infrared wavelength band are alternately arranged, as described above. In this case, the first filters that are transmission filters pass light in the blue and infrared wavelength bands. That is, the filter having a checkered pattern in which transmission filters are arranged can cope with any type of prism 12a.

Note that if any one of the image sensors 12b, 12c, and 12d has the function of further separating the wavelength band of incident light into two or more types of wavelength bands, the function need not be a filter.

According to at least one embodiment described above, it is possible to ensure image quality sufficient for the user to observe.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2019-151776, filed Aug. 22, 2019, which is hereby incorporated by reference herein in its entirety.

What is claimed is:

1. An imaging apparatus comprising:
an optical element configured to separate incident light into light components in at least three types of wavelength bands; and
a plurality of imaging elements configured to receive the light components in the at least three types of wavelength bands separated by the optical element, respectively,
wherein at least one imaging element of the plurality of imaging elements has a function of further separating a wavelength band of light that has entered the imaging element into at least two types of wavelength bands.

2. The apparatus according to claim 1, wherein the optical element separates the incident light into light in red and infrared wavelength bands, light in a green wavelength band, and light in a blue wavelength band.

3. The apparatus according to claim 2, wherein pixels of an imaging element corresponding to the green wavelength band in the plurality of imaging elements are arranged with a shift of a half pixel in at least one direction of a horizontal direction and/or a vertical direction with respect to pixels of an imaging element corresponding to the blue wavelength band.

4. The apparatus according to claim 1, wherein the optical element separates the incident light into light in a red wavelength band, light in a green wavelength band, and light in blue and infrared wavelength bands, or separates the incident light into light in the red wavelength band, light in the green and infrared wavelength bands, and light in the blue wavelength band.

5. The apparatus according to claim 1, wherein the at least one imaging element includes a filter configured to further separate the wavelength band of the light that has entered the imaging element into at least two types of wavelength bands.

6. The apparatus according to claim 5, wherein the filter includes a first filter configured to pass visible light, and a second filter configured to pass light in the infrared wavelength band.

7. The apparatus according to claim 1, wherein the at least one imaging element is a stacked imaging element.

8. An imaging apparatus comprising:
an optical element configured to separate incident light into light components in at least two types of wavelength bands; and
a plurality of imaging elements configured to receive the light components in the at least two types of wavelength bands separated by the optical element, respectively,
wherein at least one imaging element of the plurality of imaging elements has a function of further separating a wavelength band of light that has entered the imaging element into at least two types of wavelength bands, and at least one of the at least two types of wavelength bands is an infrared wavelength band.

9. The apparatus according to claim 8, wherein the optical element separates the incident light into light in red and infrared wavelength bands, and at least one of light in a green wavelength band and/or light in a blue wavelength band.

10. The apparatus according to claim 8, wherein the optical element separates the incident light into light in blue and infrared wavelength bands and at least one of light in a red wavelength band and/or light in a green wavelength band, or separates the incident light into light in the green and infrared wavelength bands and at least one of light in the red wavelength band and/or light in the blue wavelength band.

11. The apparatus according to claim 8, wherein the at least one imaging element includes a filter configured to further separate the wavelength band of the light that has entered the imaging element into at least two types of wavelength bands.

12. The apparatus according to claim 11, wherein the filter includes a first filter configured to pass visible light, and a second filter configured to pass light in the infrared wavelength band.

13. The apparatus according to claim 8, wherein the at least one imaging element is a stacked imaging element.
